(19) United States
(12) Patent Application Publication
(10) Pub. No.: US 2012/0157529 A1
(43) Pub. Date: Jun. 21, 2012

(54) Composition and Method for Prevention, Mitigation or Treatment of an Enteropathogenic Bacterial Infection

(75) Inventors: F. Jon Kull, Enfield, NH (US); Ronald K. Taylor, Lebanon, NH (US); Michael Lowden, St. Laurent (CA); Karen Skorupski, West Lebanon, NH (US); Jessica Day, Hanover, NH (US); Gabriela Kovacikova, Hanover, NH (US)

(73) Assignee: Trustees of Dartmouth College, Hanover, PA (US)

(21) Appl. No.: 13/384,860

(22) PCT Filed: Jul. 21, 2010
(86) PCT No.: PCT/US10/42704

§ 371 Date: Feb. 23, 2012

Related U.S. Application Data
(60) Provisional application No. 61/227,190, filed on Jul. 21, 2009.
(60) Provisional application No. 61/301,264, filed on Feb. 4, 2010.

(51) Int. Cl. A61K 31/201; A01P 1/00; A61P 31/04; A01N 37/06

(52) U.S. Cl. 514/560

(57) ABSTRACT

The present invention embraces the use of palmitoleic acid, or a derivative, mimetic, or extract containing the same, to decrease the expression of bacterial virulence factors thereby preventing, mitigating, or treating bacterial infection.

### INTRODUCTION

This application claims benefit of priority to U.S. Provisional Application Ser. Nos. 61/301,264, filed Feb. 4, 2010, and 61/227,190, filed Jul. 21, 2009, the contents of which are incorporated herein by reference in their entireties.

This invention was made with government support under contract numbers R01 AI060031, AI072661, AI039654 and AI41558 awarded by the National Institutes of Health. The government has certain rights in the invention.

### BACKGROUND OF THE INVENTION

The increasing resistance of bacterial pathogens to antibiotics, combined with fundamental advances in understanding the mechanisms and regulation of bacterial virulence, has prompted the identification of pathogen antivirulence drugs that antagonize the activity of virulence factors. Cholera is an acute intestinal infection caused by the bacterium Vibrio cholerae, a gram-negative flagellated bacillus. In addition to being a class B bioterrorism threat, cholera is more widespread today than it was in the previous century. The expression of V. cholerae's primary virulence factors, the toxin-coregulated pilus (TCP) and cholera toxin (CT), occurs via a transcriptional cascade involving several activator proteins, and serves as a paradigm for the regulation of bacterial virulence. Strains of V. cholerae capable of causing the significant epidemics and pandemics of cholera that have occurred throughout history possess two genetic elements, the Vibrio pathogenicity island (VPI) and the lysogenic CTX phage. Both of these elements have inserted into the circular chromosome I and are present in the pathogenic forms of the organism. The VPI contains the genes responsible for the synthesis and assembly of the essential colonization factor TCP, and the CTX phage encodes the CT genes. Expression of the TCP and CT genes is coordinately regulated at the transcriptional level by a virulence cascade involving activator proteins encoded both within the VPI and the ancestral genome. AphA and AphB initiate the expression of the cascade by a novel interaction at the tcpPH promoter. AphA is a member of a new regulator family and AphB is a LysR-type activator, one of the largest transcriptional regulatory families. Once expressed, cooperation between TcpP/TcpH and the homologous transmembrane activators ToxR/ToxS activates the toxT promoter. ToxT, an AraC/XylS (A/X) type regulator, then directly activates the promoters of the primary virulence factors. Thus, ToxT is the paramount regulator of virulence gene expression.

ToxT inhibitors have been identified and shown to provide protection against intestinal colonization by V. cholerae. For example, bile (Schuhmacher, et al. (1999) J. Bacteriol. 181:1508-14) and several of its unsaturated fatty acid constituents, i.e., oleic acid, linoleic acid, and arachidonic acid (Chatterjee, et al. (2007) Infect. Immun. 75:1946-53) have been shown to inhibit virulence factor gene expression. Similarly, virstatin, a small molecule 4-[N-(1,8-naphthalimide)]-n-butyric acid, has been shown to inhibit virulence regulation in V. cholerae (Hung, et al. (2005) Science 310(5748):670-4).

### SUMMARY OF THE INVENTION

The present invention features a method for decreasing expression of a bacterial virulence factor by contacting a pathogenic bacterium that expresses an A/X regulatory protein with a composition containing palmitoleic acid, or a derivative, mimetic, or extract containing the same.

The present invention also features a method for preventing, mitigating, or treating an infection by a bacterium that expresses an A/X regulatory protein by administering to a subject in need thereof an effective amount of a composition containing palmitoleic acid, or a derivative, mimetic, or extract containing the same.

In particular embodiments of the invention, the extract containing palmitoleic acid is an extract of Sea Buckthorn or Macadamia. In other embodiments the pathogenic bacterium is Vibrio cholerae, Escherichia coli, Shigella flexneri, Yersinia enterocolitica, Salmonella typhi, Bacillus anthracis, Listeria monocytogenes, Staphylococcus aureus or Salmonella typhimurium.

### BRIEF DESCRIPTION OF THE DRAWINGS

FIG. 1 shows binding of palmitoleate to ToxT. FIG. 1A depicts a ribbon diagram of ToxT showing α-helices, β-strands, and loops. The bound cis-palmitoleate is shown in stick form. The N- and C-termini are indicated. Helices and strands are numbered according to their topological connectivity in the full length protein. Note that residues 101-110 are disordered in the structure, as indicated by the loop ends on the left side of the molecule. FIG. 1B shows an electron density map, contoured at 1σ, around the cis-palmitoleate ligand. The side chains interacting with the ligand include Y12, K31, and K230. There is no electron density visible beyond carbon sixteen in the chain. Residues that encompass the hydrophobic pocket include Y20, F22, L25, I27, F33, L61, F69, L71, V81, V83, I226, M259, V261, Y266, and M269.

FIG. 2 shows the effects of fatty acids on tcp (FIG. 2A) and ctx (FIG. 2B) expression with a model of ToxT function. Expression is based upon β-galactosidase activity of tcpA-lacZ and ctx-lacZ fusion constructs, respectively. Cells were grown in LB medium pH 6.5 at 30° C. for 18 hours +/− the indicated fatty acids at 0.02% dissolved in methanol. C, control with methanol; PA, sodium palmitate; POA, palmitoleic acid; OA, oleic acid.

FIG. 3 is a means diamond plot. Mouse lethality data was converted to survival rates based on percentage of 48 hours at time of death. A one-way ANOVA was used to test for survival rate differences among the five groups in the challenge assay. Significant differences were observed at F(4,71)=24.8, p=0.0001. A comparison of treatment means via the post-hoc analysis Tukey HSD, p<0.05 indicated that the palmiteoleic acid (PA) and PA challenged (PAC) groups gave significantly higher survival rates than the control and methanol (MeOH) groups. Arcsine transformations were performed on the data to remove possible bias caused by percentages but there was no difference between transformed and non-transformed data. A means diamond illustrates a sample mean and 95% confidence interval. The line across each diamond represents the group mean. The vertical span of each diamond represents the 95% confidence interval for each group. Overlap marks are drawn above and below the group mean. For groups with equal sample sizes, overlapping marks indicate that the two group means are not significantly different at the 95% confidence level. The horizontal extent of each group along the x-axis (the horizontal size of the diamond) is proportional to the sample size of each level of the x variable. It follows that the narrower diamonds are usually the taller ones because fewer data points yield a less precise estimate of the group mean. Control is bacteria in broth. MeOH is methanol and MeOHC received a second dose of methanol 1 hour after infection. PA is 0.2% palmiteoleic acid dissolved in methanol and PAC includes a second dose of 0.2% palmiteoleic acid administered 1 hour after infection.

### DETAILED DESCRIPTION OF THE INVENTION

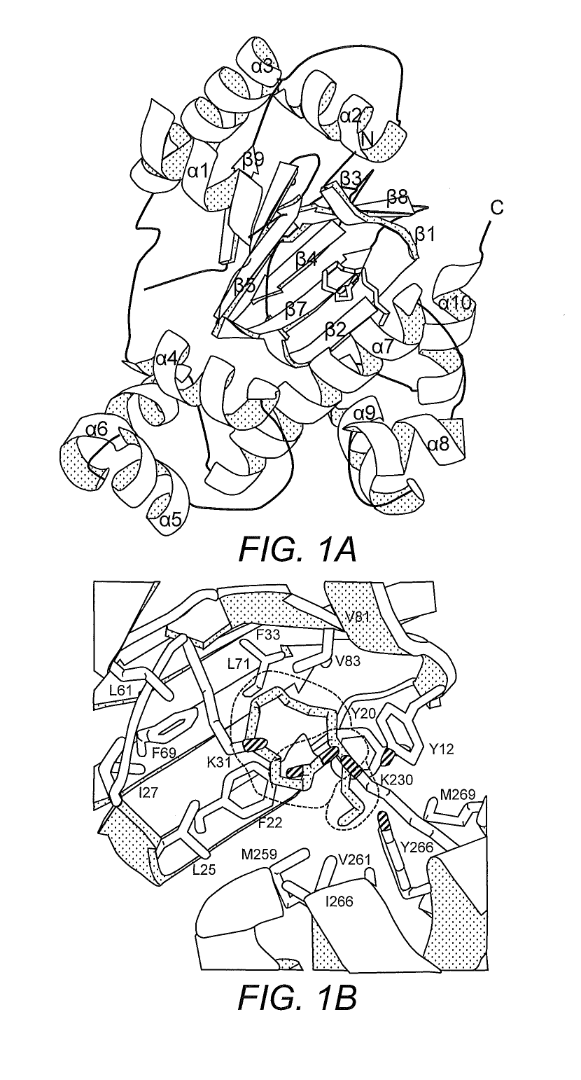

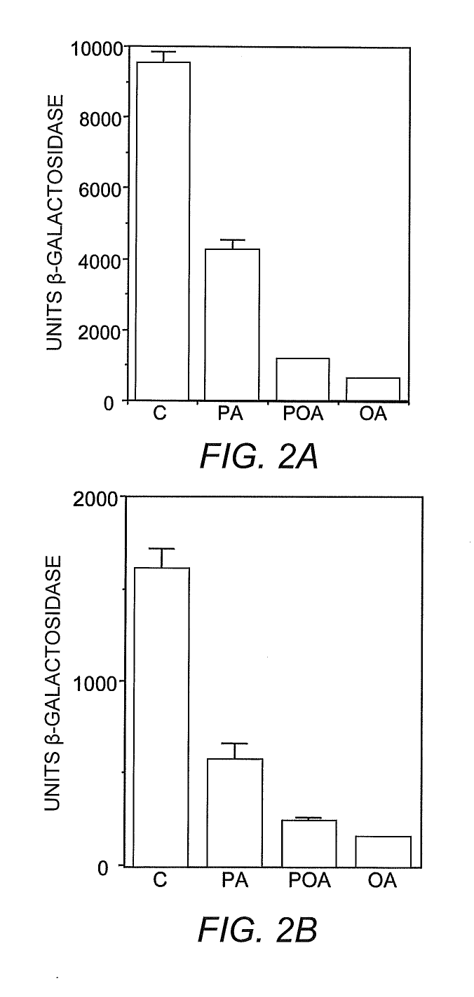

Having solved the high resolution crystal structure of ToxT of V. cholerae, it was unexpectedly found that this protein contains a monounsaturated, sixteen carbon fatty acid identified as palmitoleic acid in its deprotonated form, palmitoleate (FIG. 1). The palmitoleate appears to function to hold the ToxT in an inactive, “closed” conformation that precludes its binding to DNA and activates virulence factor genes. In vivo experiments in V. cholerae have shown that external addition of palmitoleic acid to cell culture media results in the downregulation of virulence genes tcp and ctx (FIG. 2), supporting the structural results. Indeed, the high resolution structure of ToxT demonstrates that palmitoleate binds directly to ToxT, explaining the molecular mechanism for its ability to inhibit virulence gene expression. Similarly, Sea Buckthorn oil (commercially available as a natural plant extract) also inhibits virulence gene production in in vivo experiments, albeit to a lesser degree than isolated palmitoleic and oleic acids. These findings indicate that palmitoleic acid, derivatives, mimetics, or extracts containing the same can be used to directly decrease expression of virulence genes in bacteria that express A/X regulatory proteins thereby preventing, mitigating, or treating infection by such bacteria.

Accordingly, the present invention features compositions containing palmitoleic acid, derivatives, mimetics, or extracts containing the same for use in a method for decreasing or inhibiting virulence gene expression and preventing, mitigating, or treating bacterial infection. As is conventional in the art, palmitoleic acid or (Z)-9-hexadecenoic acid, is an omega-7 monounsaturated fatty acid designated by the abbreviation 16:1Δ9. Palmitoleic acid can be obtained in an isolated form (e.g., ≧99%) from commercial sources such as Sigma-Aldrich (St. Louis, Mo.); obtained by fermentation (Xu, et al. (1999) Zhongguo Youzhi 24(6):53-5); or isolated from a variety of sources including animal oils (e.g., mink oil), vegetable oils, and marine oils (e.g., whale, seal, cod, and marine cyanobacteria, Phormidium sp. and Oscillatoria sp.; Matsunaga, et al. (1995) FEMS Microbiology Letters 133:137-141). In particular, Macadamia oil (Macadamia integrifolia) and Sea Buckthorn oil (e.g., oil from the pulp/peel and fruit of Hippophae rhamnoides) are botanical sources with high concentrations, containing 12%-39% (Yang & Kallio (2001) J. Agric. Food Chem. 49(4):1939-47) and 40% (Li & Beveridge (2003) Sea Buckthorn (Hippophae rhamnoides L.): Production and Utilization. Ottawa, Ontario: NRC Research Press. pp. 54-55) palmitoleic acid, respectively.

For the purposes of the present invention, a derivative of palmitoleic acid is a compound that has a similar structure and similar chemical properties to palmitoleic acid, but differs from it by one or more elements or groups. Examples of derivatives of palmitoleic acid include, for example, chloride, anhydride, ester or methyl ester derivatives of palmitoleic acid as well as the deprotonated form of palmitoleic acid, palmitoleate. Such derivatives can be produced using conventional methods in the art. For example, Rüsch gen. Klaas & Meurer ((2004) Euro. J. Lipid Sci. Tech. 106:412-416) describe the production of palmitoleic acid methyl ester from Sea Buckthorn juice pomace. As such, palmitoleic acid, or chloride, anhydride, ester or methyl ester derivatives of palmitoleic acid are also embraced by the invention.

Mimetics of palmitoleic acid are compounds resembling palmitoleic acid by having similar chemical or structural characteristics, which compete with binding of palmitoleate to ToxT. Such compounds can be designed and/or screened for using in silico and/or in vitro screening assays routinely employed by the skilled artisan. In this respect, molecular design techniques can be used to design, identify and synthesize mimetics capable of binding to ToxT protein and other A/X regulatory proteins. The crystal structure of ToxT (FIG. 1) can be used in conjunction with computer modeling using a docking program such as GRAM, DOCK, HOOK or AUTODOCK (Dunbrack, et al. (1997) Folding& Design 2:27-42) to identify potential mimetics that inhibit A/X regulatory protein activity. For example, molecules interacting with pocket created by beta sheets 1, 2, 3, 7 and 8 and alpha helix 7 (FIG. 1A) can be used as lead compounds for inhibitors of A/X regulatory protein activity. In particular embodiments, molecules interacting with amino acid residues Tyr12, Lys31, and Lys230 of ToxT (GenBank Accession Nos. ACP08869, ACP05115, and P0C6D6) are expected to be useful inhibitors. Further included within the scope of mimetics are cyclic compounds based on the conformation of palmitoleate observed in the crystal structure. Palmitoleate in free form is a linear molecule with a kink. But, in the context of being bound to ToxT, it folds into a U-shape with the hydrophobic tail buried in the pocket (see FIG. 1B). It is contemplated that small molecules with a shape mimicking the shape of bound palmitoleate (FIG. 1B) could bind the pocket tightly. For example, it is contemplated that cyclic, polycyclic, or heterocyclic molecules may fit into the palmitoleate binding pocket of ToxT and other A/X regulatory proteins. In vitro and/or in vivo assays can be used to detect, confirm, or monitor the inhibitory activity of a compound against these A/X regulatory proteins. Such assays include binding assays, assays detecting the expression of virulence factors, or pathogenicity assays in appropriate animal models.

Palmitoleic acid for use in the methods of the invention can also be in the form of plant, bacterial, or animal extracts. As indicated herein, Macadamia oil and Sea Buckthorn oil are botanical sources with particularly high concentrations of palmitoleic acid; 12.1-39.0% and 40%, respectively. Similarly, Phormidium sp. and Oscillatoria sp. of marine cyanobacteria have been shown to have an unusually high cis-palmitoleic acid content, 54.5% and 54.4% of total fatty acid, respectively (Matsunaga, et al. (1995) supra). Extracts of the invention can be prepared by any conventional method. See, e.g., U.S. Pat. No. 6,461,662. Such methods can include drying and/or grinding a suitable biomass source and subjecting the same to one or more solvents, thereby providing an extract, which may be either used as a crude extract or further fractionated.

Suitable methods for drying source material include: sun drying followed by a heated air-drying or freeze-drying; lyophilization or chopping the biomass into small pieces, e.g., 2-10 cm, followed by heated air-drying or freeze-drying. Once sufficient moisture has been removed, e.g., more than 90%, the material can be ground to a coarse particle size, e.g., 0.01-1 mm, using a commercial grinder.

In general terms, a suitable method for preparing an extract of the invention includes the steps of treating collected biomass material with a solvent to extract a fraction containing palmitoleic acid, separating the extraction solution from the rest of the biomass, removing the solvent from the extraction solution and recovering the extract. The extract so recovered may be further purified by way of suitable extraction or purification procedures.

More specifically, biomass material can be ground to a coarse powder as described above. Subsequently, a suitable solvent, e.g., a food grade solvent, can be added to the powder. A good grade solvent is any solvent which is suitable and approved for use in conjunction with foods intended for human consumption. Examples of suitable solvents are alcohol-based solvents, ethyl acetate, liquid carbon dioxide, hexane, and one or more components of fusel oil, e.g., ethyl acetate. Alcohol-based solvents, i.e., pure alcohol solvents and mixtures thereof with water or other organic solvents, are most desirable.

The extraction solution can then be separated from the residual biomass material by an appropriate separation procedure such as filtration and/or centrifugation. The solvent can be removed, e.g., by means of a rotary evaporator. The separated crude extract can then be tested to confirm the presence of palmitoleic acid via gas-liquid chromatography (see, e.g., Mogilevskaya, et al. (1978) Khimiko-Farmatsevticheskii Zhurnal 12:143-146, which describes chromatographic analysis of palmitoleic acid in sea buckthorn oil) or a suitable in vitro bioassay, e.g., ToxT activity assay.

Extracts of the invention can be dried to remove moisture, e.g., by spray-drying, freeze-drying or vacuum-drying, to yield a free-flowing powder. Optionally, the extracts can be dried on a pharmaceutically acceptable carrier, such as maltodextrin or starch. As yet a further alternative, biomass can be extracted and concentrated without drying to give a liquid extract, which is effective in inhibiting A/X regulatory protein activity.

Compositions of the invention can be composed of purified components (i.e., purified or isolated palmitoleic acid, derivatives, mimetics) or extracts alone, or alternatively, said compositions can contain conventional pharmaceutical or nutritionally acceptable excipients, diluents or carriers, which are used in the preparation of pharmaceuticals, nutraceuticals, nutritional compositions, such as dietary supplements, medical nutrition or functional foods. Typically, this involves mixing the active ingredients of the invention together with edible pharmaceutically or nutritionally acceptable solid or liquid carriers and/or excipients, e.g., fillers, such as cellulose, lactose, sucrose, mannitol, sorbitol, and calcium phosphates; and binders, such as starch, gelatin, tragacanth, methylcellulose and/or polyvinylpyrrolidone (PVP). Optional additives include lubricants and flow conditioners, e.g., silicic acid, silicon dioxide, talc, stearic acid, magnesium/calcium stearates and polyethylene glycol (PEG) diluents; disintegrating agents, e.g., starch, carboxymethyl starch, cross-linked PVP, agar, alginic acid and alginates, coloring agents, flavoring agents and melting agents. Dyes or pigments may be added to tablets or dragee coatings, for example, for identification purposes or to indicate different doses of active ingredient.

The composition of the invention can optionally include conventional food additives, such as any of emulsifiers, stabilizers, sweeteners, flavorings, coloring agents, preservatives, chelating agents, osmotic agents, buffers or agents for pH adjustment, acidulants, thickeners, texturizers and the like.

In addition to the above, the compositions of the present invention can further include antibiotics (e.g., tetracyclines), probiotics, prebiotics, anti-LPS sIgA (Apter, et al. (1993) Infect. Immun. 61(12):5279-5285), as well as other monounsaturated fatty acids such as oleic acid to facilitate the prevention, mitigation and/or treatment of a bacterial infection. Indeed, it is contemplated that like palmitoleic acid, other monounsaturated fatty acids will be useful in the treatment of such infections. As such, pharmaceutical compositions containing other monounsaturated fatty acids such as oleic acid and vaccenic acid and their use in the treatment of bacterial infections are also embraced by the present invention.

Suitable product formulations according to the present invention include sachets, soft gel, powders, syrups, pills, capsules, tablets, liquid drops, sublinguals, patches, suppositories, liquids, injectables and the like. Also contemplated are food and beverage products containing the composition of the present invention, such as solid food products, like bars (e.g., nutritional bars or cereal bars), powdered drinks, dairy products, breakfast cereals, muesli, candies, confectioneries, cookies, biscuits, crackers, chocolate, chewing-gum, desserts and the like; liquid comestibles, like soft drinks, juice, sports drinks, milk drinks, milk-shakes, yogurt drinks or soups, etc.

The composition of the invention can be provided as a component of a meal, e.g., a nutritional or dietary supplement, in the form of a health drink, a snack or a nutritionally fortified beverage, as well as a conventional pharmaceutical, e.g., a pill, a tablet or a softgel, for example.

Administration of the composition of the invention can be via intradermal, intramuscular, intraperitoneal, intravenous, subcutaneous, intranasal, epidural, oral, sublingual, intracerebral, transdermal, rectal, or topical administration. The mode of administration is left to the discretion of the practitioner.

Daily dosage of a composition of the present invention would usually be single or multiple servings per day, e.g., once or twice daily, for acute or chronic use. However, benefit may be derived from dosing regimens that can include consumption on a daily, weekly or monthly basis or any combination thereof. Administration of compositions of the invention, e.g., treatment, could continue over a period of days, weeks, months or years, until an infection has been treated. Optimally, the composition of the invention is consumed at least once a day on a regular basis, to prevent an infection.

ToxT belongs to the AraC/XylS (A/X) superfamily of regulatory proteins. This family is composed of approximately 1,974 members identified in 149 bacterial genomes including Bacillus anthracis, Listeria monocytogenesi and Staphylococcus aureus (Ibarra, et al. (2008) Genetica 133:65-76), and is known for its role in virulence gene regulation. Using secondary structure prediction and homology modeling, multiple candidates from the A/X protein superfamily containing lysines or other positive amino acids at positions homologous to those identified in ToxT were identified. This analysis indicated that many pathogenic bacteria, including a variety of Escherichia coli, Shigella flexneri, Yersinia enterocolitica, Salmonella typhi, and Salmonella typhimurium contain A/X regulatory proteins with homologous lysine residues and/or homologous ligand binding pockets. To demonstrate the effect of fatty acids on virulence gene production in other pathogenic bacteria, electromobility gel shift assays (EMSAs) and site-directed mutagenesis are conducted. It is expected that other pathogenic bacteria use a common, fatty acid-mediated mechanism to regulate virulence factor expression and pathogenic activity. Thus, use of compositions herein can be broadly applied to treat enteric bacterial infections that cause travelers' diarrhea, dysentery, and typhoid fever, diseases infecting some 4 billion people annually worldwide.

Thus, the present invention embraces compositions containing palmitoleic acid, derivatives, mimetics, or extracts containing the same are used in a method for decreasing or inhibiting the expression of bacterial virulence genes. This method is carried out by contacting a pathogenic bacterium with a composition of the present invention so that the expression of at least one virulence factor, e.g., TCP and/or CT in V. cholerae, is measurably decreased as compared to bacteria not contacted with the composition of the invention. A decrease or inhibition of virulence factor expression can be measured using any conventional method for monitoring nucleic acid or protein levels in a cell, e.g., northern blot analysis, RT-PCR analysis, dot blot analysis, western blot analysis and the like. Desirably, the composition of the invention decreases virulence factor expression by 10%, 20%, 30%, 40%, 50%, 60%, 70%, 80%, 90%, or as much as 100% as compared to untreated bacteria.

V. cholerae. There are several characteristics of pathogenic V. cholerae that are important determinants of the colonization process. These include adhesins, neuraminidase, motility, chemotaxis and toxin production. If the bacteria are able to survive the gastric secretions and low pH of the stomach, they are well adapted to survival in the small intestine. V. cholerae is resistant to bile salts and can penetrate the mucus layer of the small intestine, possibly aided by secretion of neuraminidase and proteases. Specific adherence of V. cholerae to the intestinal mucosa is likely mediated by the long filamentous TCP pili which are coregulated with expression of the cholera toxin genes.

As indicated herein, V. cholerae produces cholera toxin, which is composed of two A subunits and five B subunits. The B subunits allow binding to a ganglioside (GM₁) receptor on the intestinal epithelial cells. The B pentamer must bind to five corresponding GM₁ receptors. This binding occurs on lipid rafts, which anchor the toxin to the membrane for endocytosis of the A subunits, thereby trafficking the toxin into the cell and to the basolateral surface where it acts (Lencer (2001) Am. J. Physiol. Gastrointest. Liver Physiol. 280:G781-G786). Once internalized, the A subunits proteolytically cleave into A1 and A2 peptides. The A1 peptide ADP-ribosylates a GTP-binding protein, thereby preventing its inactivation. The always active G protein causes adenylate cyclase to continue forming cAMP. This increase in intracellular cAMP blocks absorption of sodium and chloride by microvilli and promotes the secretion of water from the intestinal crypt cells to preserve osmotic balance (Torgersen, et al. (2001) J. Cell Sci. 114:3737-3747). This water secretion causes the watery diarrhea with electrolyte concentrations isotonic to plasma. The fluid loss occurs in the duodenum and upper jejunum, with the ileum less affected. The colon is less sensitive to the toxin, and is therefore still able to absorb some fluid. The large volume, however, overwhelms the colon's absorptive capacity.

In addition to V. cholerae, the following is a list of some of the bacterial enteric pathogens that express A/X family members that properly align with ToxT. In so far as other pathogens may be identified based upon the structural analysis disclosed herein, the following list is merely illustrative and in no way limits the scope of bacteria that can be targeted by the instant fatty acid compositions.

Escherichia coli. There are several pathogenic derivatives of E. coli. Several of the most common are as follows. One is Enterohemorrhagic E. coli (EHEC), which causes a Shigella-like illness and is also known as the hamburger meat E. coli. Another is Enteropathogenic E. coli (EPEC), which causes persistent diarrhea in children. EPEC expresses a surface appendage termed the bundle forming pilus, or BFP. BFP is required for intestinal colonization by the bacterium. BFP gene expression is activated by the A/X family member PerA that meets alignment criteria described herein. A third example is Enterotoxigenic E. coli (ETEC), which expresses a toxin identical to ToxT and causes traveler's diarrhea. ETEC expresses colonization factor adhesions termed CS1 and CS2. The expression of the corresponding genes is activated by an A/X family regulator termed Rns that meets alignment criteria described herein. Similarly, the cof gene cluster, Longus gene cluster and CFA/I operon of ETEC also respectively encode regulatory proteins cofS, lngS and CfaD, which regulate the expression of virulence factors. Indeed, CfaD and Rns are fully interchangeable with each other (Bodero, et al. (2007) J. Bacteriol. 189:1627-32) and recognize the same DNA binding sites.

Salmonella. Salmonella cause 1.4 million cases of gastroenteritis and enteric fever per year in the US and lead all other food borne pathogens as a cause of death. While there are over a thousand serotypes of Salmonella that can cause gastroenteritis, S. enteritidis (sv. Typhimurium) is the leading cause. S. enteritidis (sv. Typhimurium) infection of mice serves as a model for typhoid fever as the causative agent of this disease only infects humans. As such, this species has served as a model organism for both gastroenteritis and typhoid fever. Most of the genes that encode virulence factors are located in clusters on salmonella pathogenicity islands termed SPIs. SPI-1 carries the genes for a type III secretion system (T3SS), the expression of which is critical for virulence. The master regulator of the expression of SPI-1 genes is HilA. The expression of HilA itself is controlled by HilD. HilD is an A/X family member that meets alignment criteria described herein.

Salmonella typhi (S. enterica sv. Typhi) is the leading cause of enteric fever also known as typhoid fever. Typhoid fever is estimated to affect approximately 17 million people annually, causing 600,000 deaths. S. typhi is a multi-organ organism, infecting lymphatic tissues, liver, spleen, and bloodstream. S. typhi has a gene regulatory network similar to the SPI-1 and regulation of T3SS gene expression in S. enteritidis (sv. Typhimurium). In the case of S. typhi the aligned A/X family member is designated SirC.

Shigella. Several Shigella species are responsible for the majority of bacillary dysentery that is caused by this organism. S. dysenteriae is common in many parts of the world. S. flexneri and S. sonnei are the most common in the U.S. Most molecular analysis regarding Shigella has been performed with S. flexneri. This species requires a surface protein, IcsA, to nucleate actin and travel through and between host cells. Expression of the icsA gene is activated by VirF, which meets alignment criteria described herein.

Bacillus anthracis. Bacillus anthracis is an aerobic spore-forming bacteria that causes anthrax disease. Livestock may become infected by eating or inhaling anthrax spores. Humans, especially farmers and individuals who work in slaughterhouses, may develop cutaneous anthrax through skin exposure to infected animals. Humans can also get inhalational anthrax by breathing in material contaminated with the bacteria. This bacterium also expresses an AraC family member.

Listeria. Listeria monocytogenes is a facultative intracellular bacterium that is the causative agent of Listeriosis. It is one of the most virulent food-borne pathogens with 20 to 30 percent of clinical infections resulting in death. Listeria monocytogenes also expresses an AraC family member.

Staphylococcus aureus. Staphylococcus aureus is a facultatively anaerobic, gram-positive coccus and is the most common cause of staph infections. Some strains of S. aureus, which produce the exotoxin TSST-1, are the causative agents of toxic shock syndrome, whereas other strains of S. aureus also produce an enterotoxin that is the causative agent of S. aureus gastroenteritis.

Yersinia enterocolitica is a common pathogen of children and adults, with a strong propensity for extraintestinal complications. Gastrointestinal disorders include enterocolitis, particularly in children, and pseudoappendicitis, particularly in young adults. Y. enterocolitica virulence factors include outer proteins termed Yops and YadA, which is an adhesin that is essential for colonization. VirF is an A/X family member that meets alignment criteria described herein.

In so far as ToxT and other A/X regulatory proteins directly regulate the expression of virulence factors, which are involved in pathogenicity, inhibition of A/X regulatory protein activity, and hence virulence factor expression, is useful in the prevention, mitigation, and/or treatment of Enteropathogenic bacterial infection. As used herein, the term “bacterial infection” is used to describe the process of adherence and virulence factor production by a pathogenic bacterium that expresses an A/X regulatory protein. For the purposes of the present invention, the term “treatment” or “treating” means any therapeutic intervention in a mammal, preferably a human or any other animal suffering from an enteropathogenic bacterial infection, such that symptoms and bacterial numbers are reduced or eliminated. By way of illustration, it is contemplated that by reducing adhesion of V. cholerae to the intestinal mucosa via TCP pili, colonization will be reduced or inhibited, thereby allowing the subject to clear the bacterial infection. “Prevention” or “preventing” refers to prophylactic treatment causing the clinical symptoms not to develop, e.g., preventing infection from occurring and/or developing to a harmful state. “Mitigation” or “mitigating” means arresting the development of clinical symptoms, e.g., stopping an ongoing infection to the degree that it is no longer harmful, or providing relief or regression of clinical symptoms, e.g., a decrease in fluid loss resulting from an infection.

According to this embodiment of the invention, a subject in need of prevention, mitigation or treatment is administered an effective amount of a composition containing palmitoleic acid or a derivative, mimetic, or extract containing the same, thereby preventing, mitigating, or treating a bacterial infection. Subjects benefiting from the method of the invention include those having (e.g., exhibiting signs or symptoms) or at risk of having (e.g., a subject exposed to a contaminated food or water source) a bacterial infection as described herein.

The terms “effective amount” means a dosage sufficient to provide prevention, mitigation and/or treatment of a bacterial infection. The amount and dosage regimen of the composition of the invention to be administered is determined in the light of various relevant factors including the purpose of administration (e.g., prevention, mitigation or treatment), the age, sex and body weight of an individual subject, and/or the severity of the subject's symptoms. In this respect, the compositions of the invention can be administered under the supervision of a medical specialist, or may be self-administered.

The invention is described in greater detail by the following non-limiting examples.

### Example 1

### Materials and Methods

ToxT Expression. ToxT was purified using the IMPACT-CN fusion protein system (New England Biolabs). Full-length ToxT was cloned from Vibrio cholerae 0395 and ligated into pTXB1 (New England Biolabs) to produce a toxT-intein/CBD (chitin binding domain) fusion construct. ToxT was expressed by autoinduction (Studier (2005) Protein Expres. Purif. 41:207-234) in ZYM-5052 media using BL21-CODONPLUS® (DE3)-RIL (Stratagene) E. coli. LB agar plates and media contained 100 μg mL⁻¹ carbenicillin and 25 μg mL⁻¹ chloramphenicol. Selenomethionine ToxT was produced by growing the same E. coli strain in a minimal medium (PASM-5052) containing a mixture of 10 μg mL⁻¹ methionine, 125 μg mL⁻¹ selenomethionine, and 100 nM vitamin B₁₂.

Purification of ToxT. Cells were harvested by centrifugation, resuspended in column buffer (20 mM Tris pH 8.0, 1 mM EDTA, and 500 mM NaCl), lysed via French press, and clarified by centrifugation. Chitin beads (New England Biolabs) were equilibrated with cold column buffer, mixed with the clarified supernatant, and incubated at 4° C. with gentle rocking. The chitin bead slurry was then loaded onto a gravity flow column, washed with 10 column volumes of column buffer, and equilibrated with five column volumes of cleavage buffer (20 mM Tris pH 8.0, 1 mM EDTA, and 150 mM NaCl). The intein with the CBD was cleaved from ToxT using cleavage buffer with 100 mM dithiothreitol (DTT) and left at 4° C. for 20 hours. Eluant from the chitin column was then loaded onto a HITRAP SP FF cationic exchange column (GE) to separate the ToxT-intein/CBD fusion protein that coeluted with the native ToxT using a sodium chloride gradient. Pure fractions were pooled and concentrated to 1.75 mg mL⁻¹ for crystallization.

Crystallization of ToxT. ToxT was crystallized in hanging drops where 50% of the drop was ToxT in buffer from the cationic exchange column, 30% of the drop was 0.1 M HEPES pH 7.5 with 10% (w/v) PEG 8000 (the mother liquor), and 20% of the drop was 36-40% 2-methyl-2,4-pentandiol (MPD) as an additive. ToxT crystals were transferred to a solution containing the mother liquor and 20% ethylene glycol as a cryoprotectant.

X-ray Data Collection. A MAD dataset from selenomethionine ToxT was collected on X6A in the National Synchotron Light Source at the Brookhaven National Laboratory, Long Island, N.Y. High resolution native data was collected on GM/CA-CAT in the Advanced Light Source at Argonne National Laboratory, Argonne, Ill. Data were indexed with XDS (Kabsch (1988) J. Appl. Crystallogr. 916-924), solved by Solve/Resolve (Terwilliger (2000) Acta Crystallogr. D 56:965-972; Terwilliger & Berendzen (1999) Acta Crystallogr. D 55:849-861), refined with CNS (Brunger (2007) Nat. Protoc. 2:2728-2733; Brunger, et al. (1998) Acta Crystallogr. D 54:905-921), and the model was built using WinCoot (Emsley & Cowtan (2004) Acta Crystallogr. D 60:2126-2132; Lohkamp, et al. (2005) CCP4 Newsletter 42). A Ramachandran plot generated with Procheck (Laskowski, et al. (1993) J. Appl. Crystallogr. 283-291; Morris, et al. (1992) Proteins 12:345-364) shows 99.6% of residues in the most favored or additionally allowed regions and no residues in the disallowed regions.

Fatty Acid Extractions. Fatty acids were extracted from samples of aqueous ToxT according to known methods (Bligh & Dyer (1959) Can. J. Biochem. Physiol. 37:911-917). Samples were resuspended in methanol-d₄ and used for NMR spectra. Positive controls of sodium palmitate (Sigma, P9767) and cis-palmitoleic acid (Fluka, 76169) were also dissolved in methanol-d₄.

NMR Experiments. All NMR experiments were acquired on a Bruker spectrometer operating at 600 MHz, utilizing a TCI cryoprobe. All data were collected at 25° C. Spectral assignment utilized chemical shift comparison with values reported in the literature for fatty acids (Gunstone, et al. (1994) The Lipid handbook (Chapman and Hall, New York), 2nd Edition) and reference spectra obtained for samples of sodium palmitate and palmitoleic acid in the same experimental conditions. The assignment was confirmed by two dimensional homonuclear NMR experiments (TOCSY (Bax & Davis (1985) J. Magn. Reson. 65:355-360), mixing times of 60 and 120 ms, and NOESY (Macura, et al. (1981) J. Magn. Reson. 43:259-281), mixing time of 200 ms), and heteronuclear. ¹H-¹³C HMQC (Muller (1979) J. Am. Chem. Soc. 101:4481-4484) and HMBC (Bax & Summers (1986) J. Am. Chem. Soc. 108:2093-2094) experiments.

Electrophoretic Mobility Shift Assays. Single-stranded, forty base-pair complimentary oligos (Operon) from the tcp promoter (5′-GTG TTA TTA AAA AAA TAA AAA AAC ACA GCA AAA AAT GAC A-3′; SEQ ID NO:1) were end labeled with a biotin-conjugated dUTP using the Biotin 3′ End Labeling Kit (Pierce) following the manufacturer's instructions and then annealed to form double-stranded fragments. EMSA's were carried out using the LightShift Chemiluminescent EMSA Kit (Pierce) following the manufacturer's instructions. Briefly, 2.5 pmole, 4 pmole, 5 pmole of ToxT were mixed with 50 fmole of double-stranded labeled DNA in a binding buffer (10 mM Tris pH 7.5, 1 mM EDTA, 100 mM KCl, 5 mM MgCl₂, 1 mM DTT, 0.3 mg mL⁻¹ BSA, 150 μg herring sperm DNA, and 10% glycerol). Fatty acids were dissolved in methanol and added to a final concentration of 0.002% using the same volume of methanol as a control. To show specificity, a 70-fold molar excess of unlabeled double-stranded tcp fragment and a 70-fold molar excess of unlabeled nonspecific DNA (42 base pairs) were added as controls. Reactions were then incubated for 30 minutes at 30° C. and loaded on a 1×TBE 6% polyacrylamide gel at 4° C. then transferred onto a positively charged membrane (HYBOND XL, GE Healthcare) and detected by chemiluminescence. An EMSA experiment was conducted using the control reagents from the LightShift Chemiluminescent EMSA Kit following the manufacturer's instructions while adding methanol and 0.02% fatty acids.

β-galactosidase Assays. β-galactosidase activity was determined by conventional methods (Miller (1972) Experiments in Molecular Genetics (Cold Spring Harbor Laboratory Press, Cold Spring Harbor, N.Y.). The tcp-lacZ and ctx-lacZ strains MBN135 (Nye, et al. (2000) J. Bacteriol. 182:4295-4303) and KSK218 (Skorupski & Taylor (1997) Proc. Natl. Acad. Sci. USA 94:265-270) were grown for 18 hours in LB media pH 6.5 at 30° C. Either methanol or the indicated fatty acids were added to 0.02%.

Immunoblot Analysis. Cell extracts from 18-hour cultures grown as for the β-galactosidase assays were prepared and analyzed on 16% SDS-polyacrylamide slab gels. Proteins were visualized by transferring to nitrocellulose and probing with anti-TcpA antibody (Sun, et al. (1991) Infect. Immun. 59:1114-118) using the ECL detection system (Amersham).

### Example 2

### Structure of Full-Length ToxT and Comparison with Other AraC-Family Members

The 1.9 Å resolution crystal structure of ToxT was solved (FIG. 1A, Table 1).

TABLE 1

| Data Collection | Native | | | | SeMet | | | |
|---|---|---|---|---|---|---|---|---|
| Space Group | P2₁2₁2₁ | | | | P2₁2₁2₁ | | | |
| Cell Dimensions | | | | | | | | |
| a, b, c (Å) | 39.3, 77.7, 83.6 | | | | 39.5, 69.5, 85.1 | | | |
| α, β, γ (°) | 90, 90, 90 | | | | 90, 90, 90 | | | |
| | | | Peak | | Inflection | | Remote | |
| Wavelength (Å) | 0.9785 | | 0.9789 | | 0.9793 | | 0.9184 | |
| Resolution Range (Å) | 19.62-1.90 | (2.00-1.90) | 19.90-2.5 | (2.60-2.50) | 19.93-2.90 | (3.00-2.90) | 19.93-2.70 | (2.80-2.70) |
| Rsym (%) | 7.5 | (41.0) | 11.4 | (65.1) | 21.7 | (56.0) | 18.3 | (61.7) |
| I/σ | 18.55 | (5.21) | 19.21 | (3.5) | 16.6 | (6.1) | 20.2 | (3.9) |
| Measured Reflections | 147129 | (20798) | 135521 | (14663) | 52574 | (5081) | 10331 | (10716) |
| Unique Reflections | 20831 | (2907) | 17810 | (2007) | 9448 | (921) | 14186 | (1480) |
| Redundancy | 7.06 | (7.15) | 7.61 | (7.31) | 5.56 | (5.52) | 7.28 | (7.24) |
| Completeness (%) | 99.8 | (100) | 99.8 | (100) | 99.6 | (100) | 99.8 | (100) |

| | |
|---|---|
| Refinement | |
| Resolution (Å) | 19.62-1.90 |
| R_{cryst}/R_{free} (%) | 21.4/24.5 |
| No. Atoms* | 2125/217 |
| R.M.S. Deviations | |
| Bond Length (Å) | 0.01 |
| Bond Angle (°) | 1.2 |
| Avg. B Factors (Å²) | 37.5 |

*Protein/solvent

The crystal contained one monomer of ToxT per asymmetric unit, with each monomer containing two domains. The N-terminal domain (amino acids 1-160) is composed of three α-helices (helix α1-α3) and a nine stranded β-sandwich (strand β1-β9) forming a “jelly roll” or “cupin-like” fold (Dunwell, et al. (2000) Microbiol. Mol. Biol. R 64:153-179) containing a binding pocket enclosed by residues Y12, Y20, F22, L25, I27, K31, F33, L61, F69, L71, V81, and V83 from the N-terminal domain and residues 1226, K₂₃₀, M259, V261, Y266, and M269 from the C-terminal domain (FIG. 1B). The volume of this predominantly hydrophobic pocket is 780.9 Å³ as calculated by the program CASTp. The pocket contains a sixteen-carbon fatty acid bound such that its negatively charged carboxylate head group forms salt bridges with both K31 from the N-terminal domain and K230 from the C-terminal domain (FIG. 1B). Following a short linker (amino acids 161-169), the C-terminal domain (170-276) is made up of two HTH DNA-binding motifs (the more N-terminal HTH1 and the more C-terminal HTH2) linked by a relatively long α-helix, helix α7. The interface between the two domains has an area of ˜2000 Å² and is very polar, with few hydrophobic interactions.

Structures of other AraC-family members are limited to three members: AraC, in which the N- (Soisson, et al. (1997) Science 276:421-425) and C-terminal (Rodgers & Schleif (2009) Proteins 77:202-208) domain structures have been determined separately, MarA, which contains only a DNA-binding domain (Rhee, et al. (1998) Proc. Natl. Acad. Sci. USA 95:10413-10418), and Rob, which, in contrast to ToxT and AraC, contains an N-terminal DNA-binding domain and a C-terminal regulatory domain (Kwon, et al. (2000) Nat. Struct. Biol. 7:424-430). Both the MarA and Rob structures have been cocrystallized with DNA. The C-terminal domain of Rob, like the N-terminal domains of ToxT and AraC, is composed of several helices and β-sheets forming a binding pocket. While the structure of Rob contains no ligand, the N-terminal domain of AraC (PDB ID 2ARC) has been determined with arabinose bound in the β-sandwich in a position similar to the fatty acid in ToxT.

A comparison of full-length ToxT with existing high resolution structures using DALI and SSM gave no significantly similar hits over the entire 276 amino acids. However, when the two domains are taken separately, the N-terminal domain of ToxT most closely resembles the N-terminal domain of AraC (for 126 α-carbons, the RMSD is 3.63 Å; PDB ID 1XJA (Weldon, et al. (2007) Proteins 66:646-654)), while the C-terminal domain is most similar to the DBD of AraC(RMSD 2.12 Å for 92 α-carbons; PDB ID 2K₉S (Rodgers & Schleif (2009) Proteins 77:202-208)). ToxT and AraC have a very similar N-terminal topology and other than the N-terminal arm of AraC (residues 7-17), all of the other secondary structural elements of these two proteins can be aligned.

### Example 3

### N-Terminal Domain

The fold of the N-terminal domain of ToxT is similar to AraC in that it contains eight antiparallel β-sheets (FIG. 1A) followed by helix α1 (Soisson, et al. (1997) Science 276:421-425). However, ToxT is missing the N-terminal arm that is present in AraC that interacts with arabinose. Helix α1 and sheet β9 are linked by a disordered region between residues 101 and 110. It has been shown that alanine substitutions of four of these residues (M103, R105, N106, and L107) show either greatly enhanced ctxAp-lacZ expression or ≦10% expression of the ctxAp-lacZ and acfA-phoA fusions (Childers, et al. (2007) J. Mol. Biol. 367:1413-1430) demonstrating this region is important for virulence gene expression. Helix α3 of ToxT is analogous to the helix that allows for coiled-coil N-terminal dimerization in the AraC structure (Soisson, et al. (1997) supra). Although ToxT is clearly a monomer in this structure and appears to bind to independent toxboxes as a monomer (Bellair & Withey (2008) J. Bacteriol. 190:7925-7931), certain promoters such as top, ctx, and tagA require ToxT dimerization on adjacent toxboxes for full activation (Bellair & Withey (2008) supra; Shakhnovich, et al. (2007) Proc. Natl. Acad. Sci. USA 104:2372-2377). In AraC, the coiled-coil is anchored at the ends by a triad of leucine residues providing stability (Soisson, et al. (1997) supra). Although analogous leucine residues are not present in α3, if ToxT were to dimerize in a manner similar to that observed in AraC, complementary salt bridges would be formed between helix α3 residues such as D141, E142, K157, and K158 of one monomer and the same residues on the other monomer. In fact, it has been suggested that a D141G substitution is able to repress msh promoters as a monomer, but is unable to activate tcp (Hsaio, et al. (2009) Infect. Immun. 77:1383-1388).

A number of residues in the N-terminal domain have been shown to be important for ToxT mediated activation of virulence gene expression (Childers, et al. (2007) supra). Those involved in maintaining an N-terminal hydrophobic core (M32, W34, I35, L42, L60, L71, W117, L127, F147-148, and F151-152) have been suggested as being essential for protein folding and stability (Childers, et al. (2007) supra). Two surface exposed glutamates, E52 in β5, and E129 in α2, as well as S140, which lies in the loop between α2 and α3, have also been shown important for function (Childers, et al. (2007) supra) for reasons not illuminated by the structure.

A small molecule inhibitor of ToxT, virstatin, has been identified (Hung, et al. (2005) Science 310:670-4) that interferes with ToxT's ability to dimerize and activate transcription of the tcp and ctx promoters (Shakhnovich, et al. (2007) supra). It was also demonstrated that a L114P substitution is virstatin resistant, suggesting that it may favor a conformation that allows the protein to dimerize more efficiently (Shakhnovich, et al. (2007) supra). It is of note that L114 lies in the vicinity of the unresolved residues (residues 101 and 110) (FIG. 1A) and substitution to a proline may result in a conformational change affecting the adjacent unresolved loop or N-terminal ligand binding pocket.

### Example 4

### DNA-Binding Domain

The DBD of ToxT is composed of seven α-helices. HTH1 is composed of α5 and α6, HTH2 is composed of α8 and α9, and they are connected by a central helix α7 (FIG. 1A). Helix α4 and helix α10 are involved in scaffolding and stability of HTH1 and HTH2, respectively. Pair-wise SSM alignments performed by WinCoot of the DBD's of ToxT (amino acids 170-273), AraC, and MarA, show consistently close alignments of HTH2, with greater variability in the orientation of HTH1. The DNA-bound structure of MarA demonstrates that it is possible for AraC-family members to utilize helices α6 and α9, oriented in a parallel manner, to bind consecutive major grooves on curved target DNA (Rhee, et al. (1998) supra). This parallel arrangement is conserved in Rob; however the structure does not show both HTH motifs bound to major grooves (Kwon, et al. (2000) supra). As has been suggested (Rodgers & Schleif (2009) supra), helix α6 of AraC, which is at a divergent angle with respect to helix α9, would likely have to undergo a conformational change in order to allow for consecutive major groove binding on target DNA. In ToxT, helix α6 is not only nonparallel with helix α9, but is also more distorted and bent when compared to what is observed in AraC. Another difference in this domain is in the orientation of helix α7. In AraC and MarA, the orientation of helix α7 is virtually the same, whereas in ToxT helix α7 is orientated differently with respect to the other structures. As discussed herein, the position of helix α7 is such that it could link the N-terminal binding pocket to conformational changes occurring in the DNA-binding domain.

Residues identified in the C-terminal domain as being important for ToxT function include several in the cores of HTH1 and HTH2 (I174, V178, W186, W188, L206, V211, I217, F245, F251, and F255) (Childers, et al. (2007) supra), which are critical for proper folding and stability. There are also a number of surface exposed residues that could be involved in stabilizing the DBD (S175, R184, R221, 5227, E233, K237, G244, and N260) (Childers, et al. (2007) supra). Furthermore, it appears that residues such as K203 (α6), R214 (α7), T253 (α9), and S257 (α9) are positioned to be directly involved in protein/DNA interactions.

### Example 5

### A Fatty Acid is Present in ToxT and Influences its DNA-Binding Activity

Unsaturated fatty acids (UFAs) such as arachidonic, linoleic, and oleic acid have been shown to strongly inhibit the expression of ToxT-activated genes, whereas saturated fatty acids (SFAs) such as palmitic and stearic acid were not shown to inhibit the expression of ToxT-activated genes (Chatterjee, et al. (2007) supra). The structure of ToxT contains an almost completely buried and solvent inaccessible sixteen-carbon fatty acid bound to the pocket in the N-terminal domain (FIGS. 1A and 1B). The negative charge on the carboxylate head group hydrogen bonds with Y12 and forms salt bridges with K31 from the N-terminal domain and K230 from the C-terminal domain (FIG. 1B). NMR studies of chloroform/methanol extractions from pure ToxT samples indicate the presence of a long-chain, singly unsaturated fatty acid in a cis configuration. Although the electron density ends after carbon sixteen of the hydrophobic chain, indicating cis-palmitoleate, the ToxT structure could accommodate the two additional carbons of oleate. An Fₒ-F_{c} difference map calculated after refinement with oleate placed into the pocket shows strong negative density after carbon sixteen, further indicating that the bound molecule is cis-palmitoleate.

To address whether cis-palmitoleate was capable of influencing the activity of ToxT, different UFAs and SFAs were added to cultures of V. cholerae strains carrying transcriptional fusions to the tcp and ctx operons. It was observed that the expression of these operons were reduced between 6-8 fold with cis-palmitoleic acid and between 10-15 fold with oleic acid, whereas a two-fold reduction was observed with palmitic acid (FIGS. 2A and 2B). As previous studies have shown that toxT transcription is unaffected by UFAs, it has been suggested that UFAs act on ToxT directly (Chatterjee, et al. (2007) supra).

EMSA were performed, and a 100-fold molar excess of protein was shown to bind to a 40 base-pair probe containing two toxboxes from the tcp promoter in vitro. This interaction is specific since it was completely inhibited by a 70-fold molar excess of specific competitor DNA, but not by a 70-fold molar excess of nonspecific competitor DNA. Addition of methanol or 0.002% palmitic acid to the reaction had no effect on ToxT binding. However, addition of 0.002% palmitoleic or oleic acid completely prevented ToxT from binding to DNA, consistent with the reduction of tcp and ctx transcription observed in the presence of these fatty acids. A control EMSA experiment with a different protein/DNA pair was also performed to show that unsaturated fatty acids do not block all protein/DNA interactions.

As no fatty acids were added to any buffer or crystallization condition, the cis-palmitoleate most likely originated from the E. coli used as the protein expression strain. Indeed, cis-palmitoleic acid comprises 10.5% of the total fatty acid content in E. coli membranes, whereas oleic acid is absent (Oldham, et al. (2001) Chem. Senses 26:529-531). As it is expected that there would be very little free fatty acid in the cytoplasm of these bacteria, it is likely that ToxT bound cis-palmitoleate released from the membrane upon cell lysis. Similar phenomena have been observed such as the binding of cis-vaccenic acid by the pheromone-binding protein of Bombyx mori when purified from an E. coli expression system (Oldham, et al. (2001) supra). Previous studies indicate that 23.5% of the fatty acid content of bile is oleic acid, and if cis-palmitoleic acid is present in bile, it is at a concentration of less than 0.5%. As both oleic and cis-palmitoleic acids are monounsaturated at the ninth carbon and as there is room in the ToxT structure to potentially accommodate the longer oleic acid, it is not surprising that both fatty acids can serve as a ligand for ToxT. However, given the abundance of oleic acid in bile when compared to cis-palmitoleic acid, oleic acid may be the natural ligand responsible for altering ToxT function in vivo.

### Example 6

### A Structural Model for ToxT Activation

The finding that UFAs reduce the expression of tcp and ctx expression in V. cholerae and that they significantly reduce the ability of ToxT to bind to DNA in vitro indicates a model for the regulation of ToxT function via fatty acid binding. In this model, when the bacteria are in the lumen of the intestine in the presence of fatty acids, the position of the carboxylate head group of the fatty acid bridging K31 from the N-terminal domain with K230 from the C-terminal domain (FIG. 1B) keeps ToxT in a “closed” conformation that is not capable of binding DNA (Yu & DiRita (1999) J. Bacteriol. 181:2584-2592). Restraint of K230, which is located at the C-terminal end of helix α7, would cause helix α7 to assume a position that pulls and distorts helix α6 into an orientation that is unfavorable for DNA-binding. Once the bacteria have penetrated the mucus of the intestine where the concentrations of fatty acids are presumably reduced (Schulmacher & Klose (1999) J. Bacteriol. 181:1508-1514), charge-charge repulsion between K31 and K230 destabilizes the closed conformation, leading to an opening of the N- and C-terminal domains. In this “open” conformation, K230, helix α7, and helix α6 would no longer be restrained, and reorient into a conformation that is competent for DNA-binding. The EMSA data support this model, in which an equilibrium exists between fatty acid bound “closed” ToxT that cannot bind to DNA and fatty acid free “open” ToxT that can bind to DNA. While a 50-fold molar excess of ToxT over the probe is not sufficient to drive the binding equilibrium in the direction of the DNA-bound state, increasing the concentration of ToxT shifts the equilibrium in the direction of a protein/DNA complex. Addition of 0.002% palmitoleic or oleic acid then disrupts the protein/DNA complex by shifting the equilibrium back to the “closed” state, containing a protein/fatty acid complex, releasing it from DNA. As discussed herein, a number of studies have suggested that ToxT dimerizes upon binding to adjacent toxboxes (Withey & DiRita (2006) Mol. Microbiol. 59:1779-1789; Shakhnovich, et al. (2007) supra; Hung, et al. (2005) supra; Prouty, et al. (2005) Mol. Microbiol. 58:1143-1156). It is clear from the structure that side-by-side dimerization of “closed” ToxT on adjacent toxboxes would be difficult if not impossible due to steric constraints. However, it is expected the “open” form of ToxT would be able to dimerize on adjacent toxboxes in either the direct or inverted orientations.

### Example 7

### Palmiteoleic Acid Prevents Cholera in an Infant Mouse Model

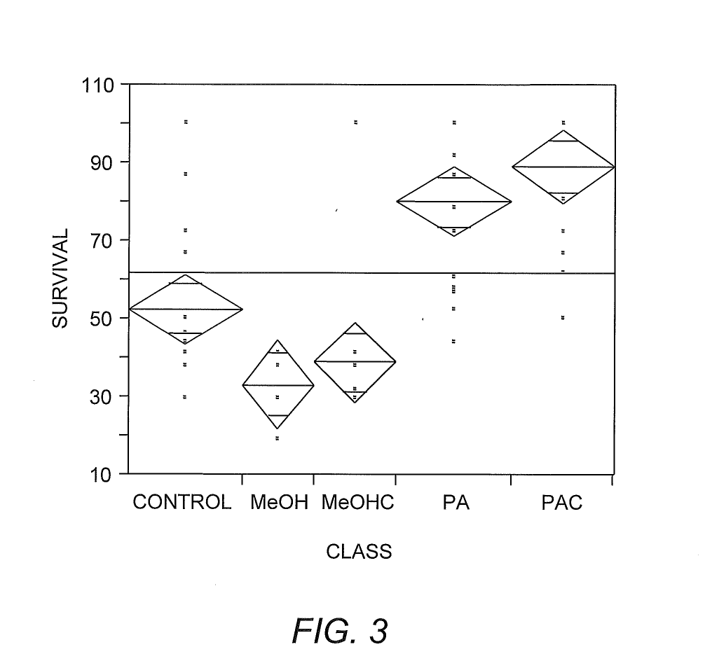

To demonstrate the feasibility of using unsaturated fatty acids to prevent cholera, palmiteoleic acid was tested in a well-established infant mouse cholera model. Palmiteoleic acid (0.2%) dissolved in methanol was co-administered orally with approximately a 10 lethal dose 50 (LD₅₀) of V. cholerae strain 0395. In some cases an additional administration of palmiteoleic acid was given to the mice one hour after infection. The ability of palmiteoleic acid to prevent death from cholera was assessed at 48 hours post-infection. The results are presented in FIG. 3 as a means diamond plot. A summary of the p values derived from the diamond plot is listed in Table 2.

TABLE 2

| | Tukey HSD test | | | | |
|---|---|---|---|---|---|
| | Variable: Survival (LD₅₀ FA challenge minus OA) | | | | |
| | {1} | {2} | {3} | {4} | {5} |
| | M = | M = | M = | M = | M = |
| Class | 52.721 | 33.335 | 39.024 | 80.093 | 89.389 |
| Control {1} | | 0.065819 | 0.278018 | 0.000524* | 0.000126* |
| MeOH {2} | 0.065819 | | 0.947113 | 0.000125* | 0.000125* |
| MeOHC {3} | 0.278018 | 0.947113 | | 0.000125* | 0.000125* |
| PA {4} | 0.000524* | 0.000125* | 0.000125* | | 0.606961 |
| PAC {5} | 0.000126* | 0.000125* | 0.000125* | 0.606961 | |

*Differences were significant at p < 0.05.

The combined results demonstrate that palmiteoleic acid was extremely effective at protecting the mice from cholera. The data was very robust with p values well below the cutoff for significance of 0.05.

What is claimed is:

1. A method for decreasing expression of a bacterial virulence factor comprising contacting a bacterium that expresses an A/X regulatory protein with a composition containing palmitoleic acid, or a derivative, mimetic, or extract containing the same, so that the expression of a virulence factor by said bacterium is decreased.

2. The method of claim 1, wherein the extract containing palmitoleic acid is an extract of Sea Buckthorn or Macadamia.

3. The method of claim 1, wherein the bacterium is Vibrio cholerae, Escherichia coli, Shigella flexneri, Yersinia enterocolitica, Salmonella typhi, Bacillus anthracis, Listeria monocytogenes, Staphylococcus aureus or Salmonella typhimurium.

4. A method for preventing, mitigating, or treating an infection by a bacterium that expresses an A/X regulatory protein comprising administering to a subject in need thereof an effective amount of a composition containing palmitoleic acid, or a derivative, mimetic, or extract containing the same, so that an infection by a bacterium that expresses an A/X regulatory protein is prevented, mitigated, or treated.

5. The method of claim 4, wherein the extract containing palmitoleic acid is an extract of Sea Buckthorn or Macadamia.

6. The method of claim 4, wherein the bacterium is Vibrio cholerae, Escherichia coli, Shigella flexneri, Yersinia enterocolitica, Salmonella typhi, Bacillus anthracis, Listeria monocytogenes, Staphylococcus aureus or Salmonella typhimurium.


SEQUENCE LISTING (1 sequence)

SEQ ID NO: 1 - DNA, 40 bp, Artificial Sequence
  Synthetic oligonucleotide
gtgttattaaaaaaataaaaaaacacagcaaaaaatgaca